(19) United States
(12) Patent Application Publication
(10) Pub. No.: US 2018/0028436 A1
(43) Pub. Date: Feb. 1, 2018

(54) TOPICAL FORMULATION OF HOPS EXTRACT

(71) Applicants: Fatemeh Abdi, Tehran (IR); Fahimeh Ramezani Tehrani, Tehran (IR); Hamid Mobedi, Tehran (IR)

(72) Inventors: Fatemeh Abdi, Tehran (IR); Fahimeh Ramezani Tehrani, Tehran (IR); Hamid Mobedi, Tehran (IR)

(21) Appl. No.: 15/727,580

(22) Filed: Oct. 7, 2017

Related U.S. Application Data
(60) Provisional application No. 62/408,705, filed on Oct. 15, 2016.

(51) Int. Cl. A61K 8/97; A61K 47/40; C12C 3/00; A61K 9/06; A61Q 90/00; A61K 8/73; A61K 9/00

(52) U.S. Cl. CPC A61K 8/97; A61K 8/738; A61K 47/40; A61K 9/0014; A61K 9/06; A61Q 90/00; C12C 3/00

(57) ABSTRACT

Disclosed herein is a method for reducing menopausal symptoms. The method may comprise preparing a topical formulation of hops extract, and administering the topical formulation on to a part of skin. Moreover, preparing the topical formulation of hops extract may include preparing the hops extract, encapsulating the hops extract in cyclodextrin to form hops nanoparticles, and dispersing the hops nanoparticles with a concentration between 5 and 15 percent by weight of the formulation in an ointment-based excipient to form the topical formulation of hops extract.

### CROSS-REFERENCE TO RELATED APPLICATION

This application claims the benefit of priority from pending U.S. Provisional Patent Application Ser. No. 62/408,705, filed on Oct. 15, 2016, and entitled “Ointment Containing Hops Nanoparticles for the Treatment of Menopausal Symptoms,” which is incorporated herein by reference in its entirety.

### TECHNICAL FIELD

The present disclosure generally relates to a method for reducing menopausal symptoms. The present disclosure further relates to a topical formulation of hops extract which includes hops nanoparticles.

### BACKGROUND

Menopause is a critical stage with a variety of symptoms in every woman's life. Menopausal symptoms, which result from estrogen deficiency during menopause, can have a significant negative impact on women's health and may increase the risks for both morbidity and mortality such as increased cognitive changes and osteoporosis.

Therefore, there is a need for menopausal symptoms to be controlled and reduced. Administration of exogenous estrogen in a hormone replacement therapy (HRT) has been a common treatment for menopausal symptoms. However, HRT can increase the risk of various other health issues, such as breast cancer, endometrial cancer, thromboembolism disorders, stroke, and coronary heart disease. Concerns about the safety of hormone replacement therapy have raised interest in use of phytoestrogens, which are naturally-occurring plant nutrients that exerts an estrogen-like impact on the body, as an alternative method for treatment of menopausal symptoms. There are many phytoestrogen sources, for example, soy beans, sesame seeds, wheat, berries, oats, barley, and hops extract.

Oral or injectable administration of phytoestrogens faces some difficulties, such as hepatic drug metabolism, destruction of drug by the digestive system, and low patient compliance. Therefore, there is a need in the art to provide a formulation of phytoestrogens in herbal treatments of menopausal symptoms with high bioavailability, no hepatic drug metabolism, controlled release of drug, and easy application.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a method for reducing menopausal symptoms. The method may comprise preparing a topical formulation of hops extract, and administering the topical formulation onto a part of skin. Moreover, preparing the topical formulation may comprise preparing the hops extract, encapsulating the hops extract in cyclodextrin to form hops nanoparticles, and dispersing the hops nanoparticles in an ointment-based excipient to form the topical formulation of hops extract.

According to an exemplary embodiment, the hops nanoparticles may be dispersed in the ointment-based excipient with a concentration between about 5 and about 15 percent by weight of the formulation. In an exemplary embodiment, the hops nanoparticles may have an average size between 80 nm and 180 nm.

According to an exemplary embodiment, preparing hops extract may include drying a plurality of hops flowers to form hops flower powder, and incubating hops flower powder with ethanol to obtain the hops extract.

According to some exemplary implementations, encapsulating the hops extract in the cyclodextrin may include dissolving a plurality of hops extract powder in a protic solvent to form a hops extract solution, dissolving the cyclodextrin in water to form a cyclodextrin solution, mixing the hops extract solution with the cyclodextrin solution to form a suspension of hops nanoparticles, and drying the suspension of hops nanoparticles to form hops nanoparticles.

According to some exemplary embodiments, the cyclodextrin may include β-cyclodextrin. In an exemplary embodiment, the protic solvent may include a water-miscible solvent including water, ethanol, or combinations thereof. In an exemplary embodiment, the ointment-based excipient may include one of Vaseline, or paraffin, or combinations thereof.

According to an exemplary embodiment, dispersing the hops nanoparticles in the ointment-based excipient may be done using one of a homogenizer, a stirrer, an agitator, a sonicator, an ultrasound device, or combinations thereof.

According to some exemplary implementations, administering the topical formulation may comprise topically administering the topical formulation with a therapeutically effective amount of hops nanoparticles for reducing menopausal symptoms. In an exemplary embodiment, the therapeutically effective amount of the hops nanoparticles may be between about 30 mg/day and about 90 mg/day. Moreover, the menopausal symptoms may include one of hot flushes, night sweats, sleep disturbances, fatigue, anxiety, sexual problems, and dryness of vagina, or combinations thereof.

In another general aspect, the present disclosure describes a topical formulation for reducing menopausal symptoms. The topical formulation may include a plurality of hops nanoparticles and an ointment-based excipient, and the hops nanoparticles with a concentration between about 5 and about 15 percent by weight of the formulation may be dispersed within the ointment-based excipient. Moreover, each hops nanoparticle may include hops extract which may be encapsulated in cyclodextrin.

According to some exemplary embodiments, the hops nanoparticles may have an average size between about 80 nm and about 180 nm. Moreover, the hops nanoparticles may have a pH between about 5 and about 7.

In some exemplary implementations, the cyclodextrin may include β-cyclodextrin matrix. The ointment-based excipient may include Vaseline, or paraffin, or combinations thereof.

According to some exemplary implementations, the topical formulation may be topically administered with a therapeutically effective amount of hops nanoparticles for reducing menopausal symptoms. In an exemplary embodiment, the therapeutically effective amount of the hops nanoparticles may be between about 30 mg/day and about 90 mg/day. Moreover, the menopausal symptoms may include one of hot flushes, night sweats, sleep disturbances, fatigue, anxiety, sexual problems, and dryness of vagina, or combinations thereof.

Other systems, methods, features and advantages of the exemplary embodiments will be, or will become, apparent to one of ordinary skill in the art upon examination of the following figures and the accompanying detailed description. It is intended that all such additional systems, methods, features and advantages be included within this description and this summary, be within the scope of the consistent with exemplary embodiments of the present disclosure, and be protected by the following claims.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a method for reducing menopausal symptoms, consistent with an exemplary embodiment of the present disclosure.

FIG. 1B illustrates a method for encapsulating hops nanoparticles in cyclodextrin, consistent with an exemplary embodiment of the present disclosure.

FIG. 2 illustrates a schematic of a topical formulation, consistent with an exemplary embodiment of the present disclosure.

FIG. 3A illustrates a transmission electron microscopy (TEM) image of hops nanoparticles, consistent with an exemplary embodiment of the present disclosure.

FIG. 3B illustrates a magnified transmission electron microscopy (TEM) image of hops nanoparticles, consistent with an exemplary embodiment of the present disclosure.

FIG. 4 illustrates a size distribution curve of hops nanoparticles, consistent with an exemplary embodiment of the present disclosure.

FIG. 5 illustrates a zeta potential curve of hops nanoparticles, consistent with an exemplary embodiment of the present disclosure.

FIG. 6A illustrates a MTT plot of the fibroblast cells after 24 hours of exposing to the hops nanoparticles, consistent with an exemplary embodiment of the present disclosure.

FIG. 6B illustrates a MTT plot of the fibroblast cells after 72 hours of exposure to the hops nanoparticles, consistent with an exemplary embodiment of the present disclosure.

### DETAILED DESCRIPTION

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Many herbal treatments for reducing menopausal symptoms involve using different active compounds such as 8-prenylnaringenin, 6-PN, isoxanthohumol, 2-methyl-3-buten-2 ol, and xanthohumol, which are examples of phytoestrogen compounds. Extract of hops flower also contains potent phytoestrogen compounds, such as 8-prenylnaringenin which is eight times stronger than other known phytoestrogens. The phytoestrogen 8-prenylnaringenin has an ability to reduce menopausal symptoms by reducing serum-luteinizing hormone (LH) and follicle-stimulating hormone (FSH), increasing uterine weight, and inducing vaginal secretory epithelium.

Disclosed herein is a method for reducing menopausal symptoms. The method may include the steps of preparing a topical formulation of hops extract, and administering the topical formulation onto a part of skin. The topical formulation of hops extract may include different active components such as 8-prenylnaringenin. Moreover, the topical formulation of the present disclosure may include a plurality of hops nanoparticles which may be dispersed within an ointment-based excipient.

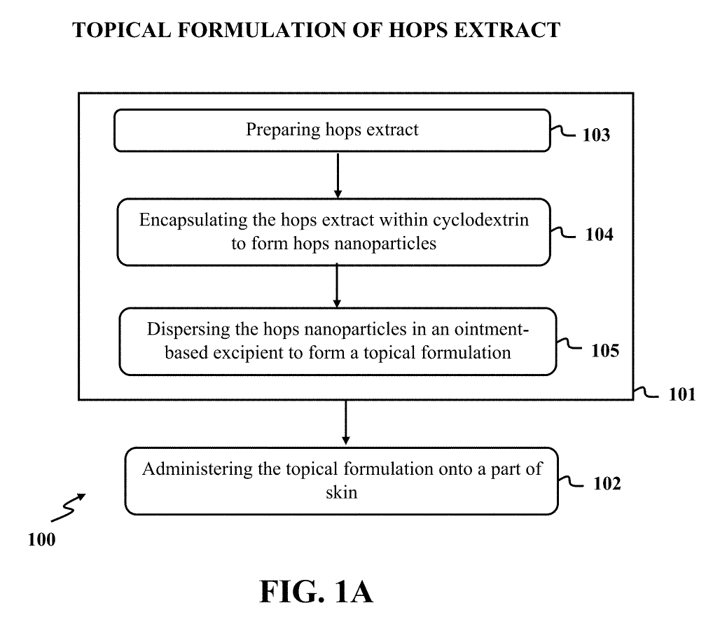

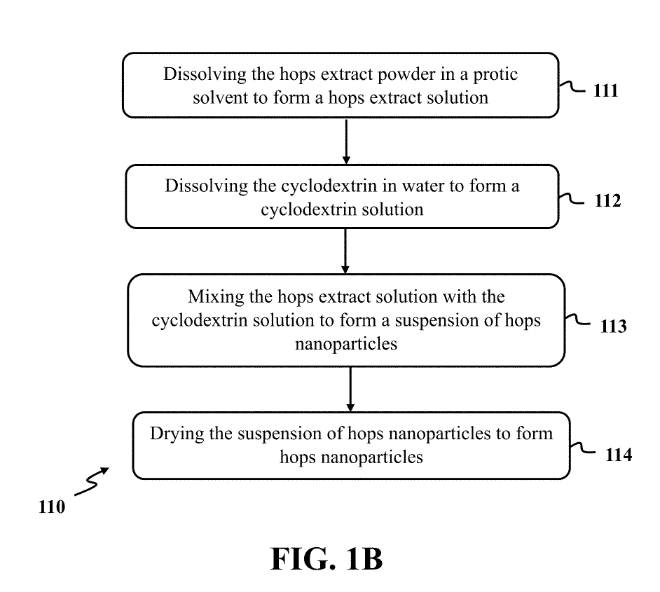

FIG. 1 is a method 100 for reducing menopausal symptoms, consistent with an exemplary embodiment of the present disclosure. The method 100 may include preparing a topical formulation of hops extract (step 101), and administering the topical formulation onto a part of skin (step 102). Preparing the topical formulation of hops extract (step 101) may include preparing hops extract (step 103), encapsulating the hops extract in cyclodextrin to form hops nanoparticles (step 104), and dispersing the hops nanoparticles in an ointment-based excipient to form a topical formulation (step 105.)

In step 103, the hops extract may be prepared via a procedure including drying a plurality of hops flowers to form hops flower powder, and incubating the hops flower powder with a solvent to obtain the hops extract. In an exemplary embodiment, the solvent may be a hydro alcoholic solvent, which may include water, or ethanol, or methanol, or combinations thereof.

In an exemplary implementation, the hops flower powder may be incubated with the solvent for a period of time. In embodiments, it may be for one day or more. After incubation, the obtained hops extract may be filtered to remove the insoluble and large particles from the hops extract. Moreover, the hops extract powder may be obtained by drying the hops extract.

Step 104 may include encapsulating the hops extract in the cyclodextrin to form the hops nanoparticles. Encapsulating the hops nanoparticles in the cyclodextrin may be done utilizing the procedure described in method 110 of FIG. 1B.

FIG. 1B illustrates a method 110 for encapsulating hops nanoparticles in cyclodextrin, consistent with an exemplary embodiment of the present disclosure. The method 110 may include dissolving the hops extract powder in a protic solvent to form a hops extract solution (step 111), dissolving the cyclodextrin in water to form a cyclodextrin solution (step 112), mixing the hops extract solution with the cyclodextrin solution to form a suspension of hops nanoparticles (step 113), and drying the suspension of hops nanoparticles to form hops nanoparticles (step 114.)

Step 111 may include dissolving the hops extract powder in a protic solvent to form a hops extract solution. In an exemplary embodiment, the hops extract solution may have a pH between about 4.2 and about 5.6. Moreover, the protic solvent may be a water-miscible solvent including water, ethanol, or combinations thereof.

Step 112 may include dissolving the cyclodextrin in water to form a cyclodextrin solution. In some exemplary embodiments, the cyclodextrin may include β-cyclodextrin matrix. In some exemplary embodiments, the cyclodextrin solution may have a pH of about 7.

Step 113 may include mixing the hops extract solution with the cyclodextrin solution to form a suspension of hops nanoparticles. The hops extract solution may be mixed with the cyclodextrin solution to form a suspension of hops nanoparticles with a pH about 5.1.

Mixing the hops extract solution and the cyclodextrin solution mat be done using one of a homogenizer, a stirrer, an agitator, a sonicator, an ultrasound device, or combinations thereof. The suspension of hops nanoparticles may be formed through encapsulating the hops extract in the β-cyclodextrin matrix as the carrier.

In some exemplary embodiments, in the encapsulation process, the hydroxyl groups of β-cyclodextrin matrix chemically interact with methoxy groups of the hops extract through a host-guest interaction. The host-guest interaction may result from electrostatic forces, Van der Waals forces, hydrophobic reactions, hydrogen bonds, structural tension release, and charge transfer interactions between the hydroxyl groups of β-cyclodextrin matrix and methoxy groups of the hops extract.

In exemplary embodiments, encapsulating the hops extract in the cyclodextrin may lead to a formulation with higher solubility, improved dermal penetration, and a controlled release of the hops extract. Furthermore, half-life of the hops extract may be increased in an encapsulated form.

Step 114 may include drying the suspension of hops nanoparticles to form hops nanoparticles. The suspension of hops nanoparticles may be dried to form hops nanoparticles. Drying the suspension of hops nanoparticles may be done using a freeze-dryer device, which works by freezing the suspension of hops nanoparticles and then reducing the surrounding pressure to allow the frozen solvents in the suspension of hops nanoparticles to sublime directly from the solid phase to the gas phase. After that, hops nanoparticles may be formed by drying the suspension of hops nanoparticles.

Then getting back to method 100 of FIG. 1A, step 105 may include dispersing the hops nanoparticles in an ointment-based excipient to form a topical formulation of hops extract. The hops nanoparticles may be dispersed in an ointment-based excipient for about 1 hours to form the topical formulation.

Dispersing the hops nanoparticles in the ointment-based excipient may be done utilizing a homogenizer, a stirrer, an agitator, a sonicator, an ultrasound device, or combinations thereof. The excipient may be an ointment-based excipient which may include Vaseline, paraffin, or combinations thereof. The hops nanoparticles may be dispersed within the ointment-based excipient with a concentration between 5 and 15 percent by weight of the formulation to form a topical formulation.

Step 102 may include administering the topical formulation on to a part of skin. The topical formulation may be topically administered onto a part of skin, for example arm skin, for reducing menopausal symptoms. The menopausal symptoms may include hot flushes, night sweats, sleep disturbances, fatigue, anxiety, sexual problems, and dryness of vagina. The therapeutically effective amount of the hops nanoparticles may be between about 30 mg/day and 90 mg/day.

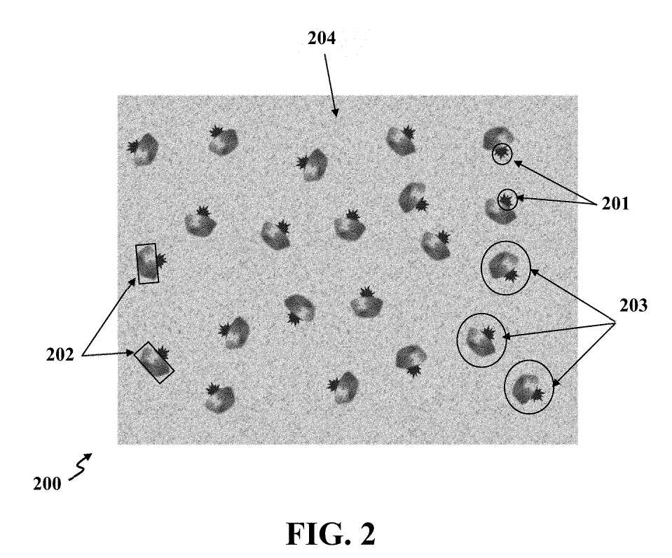

FIG. 2 illustrates a schematic of topical formulation 200, consistent with an exemplary embodiment of the present disclosure. The topical formulation 200 may include a plurality of hops nanoparticles 203 and an ointment-based excipient 204. The hops nanoparticles 203 may be dispersed within the ointment-based excipient 204.

According to some exemplary embodiments, each hops nanoparticle 203 may include hops extract 201 which may be encapsulated in cyclodextrin 202. The hops nanoparticles 203 may be present in the topical formulation 200 with a concentration between about 5 percent by weight of the formulation and about 15 percent by weight of the formulation.

According to some exemplary embodiments, the hops nanoparticles 203 may have an average size between about 80 nm and about 180 nm. The hops nanoparticles 203 may have a pH between about 5 and about 7. Moreover, the ointment-based excipient 204 may include Vaseline, paraffin, or combinations thereof. In some implementations, the cyclodextrin 202 may include β-cyclodextrin matrix.

According to an exemplary embodiment, the topical formulation 200 may be topically administered onto a part of skin with a therapeutically effective amount of hops nanoparticles 203 for reducing menopausal symptoms. The therapeutically effective amount of the hops nanoparticles 203 may be between about 30 mg/day and about 90 mg/day.

### EXAMPLES

The following examples describe exemplary embodiments of preparation method of topical formulation of the present disclosure. Furthermore, physicochemical properties of hops nanoparticles, toxicity assay, in-vivo studies, and clinical studies of topical formulation are examined.

### Example 1: Preparation of a Topical Formulation of Hops Extract

In this example, a topical formulation of hops extract was prepared in following steps: preparing hops extract, encapsulation of the hops extract in cyclodextrin as a carrier to form hops nanoparticles, and dispersing the hops nanoparticles in an ointment-based excipient to form a topical formulation.

At first, the hops extract was prepared as follows. The hops flowers were dried, and the dried hops flowers were powdered. After powdering the dried flowers, the hops flower powder was placed inside a container; then, the hops extract was obtained in a percolation method as follows. The percolation method is an extraction process that involves the slow descent of a solvent through a powdered substance until it absorbs certain constituents and drips out through the filtered bottom of the container.

In the next step, the dried flowers were mixed with ethanol 60:40 (ethanol:water) in a concentration ratio of about 1:7 (weight/volume) (hops flower powder:ethanol) to form a mixture. After that, the mixture was incubated at room temperature for about 72 hours to obtain the hops extract.

The obtained hops extract was filtered to remove the insoluble and large particles from the hops extract. Moreover, for preparing hops extract powder, the obtained hops extract was dried by using a freeze-dryer device, which removes the solvents from the hops extract. The extract powder was stored in glass containers a temperature less than about 4° C. for future use.

In next step, the hops extract powder was encapsulated in β-cyclodextrin as a carrier to form hops nanoparticles as follows. The hops extract powder was dissolved in ethanol as a protic solvent to form a hops extract solution. Also, a plurality of β-cyclodextrin was dissolved in water to form a cyclodextrin solution with a pH about 7.

After that, a suspension of hops nanoparticles was prepared through mixing the hops extract solution and the cyclodextrin solution using a magnet stirrer. Therefore, the suspension of hops nanoparticles with a pH about 5.1 were formed through encapsulation of the hops extract in the matrix of β-cyclodextrin as the carrier.

In the encapsulation process, the hydroxyl groups of β-cyclodextrin matrix chemically interact with methoxy groups of the hops extract through a host-guest interaction. The host-guest interaction may result from electrostatic forces, Van der Waals forces, hydrophobic reactions, hydrogen bonds, structural tension release, and charge transfer interactions between the hydroxyl groups of β-cyclodextrin matrix and methoxy groups of the hops extract.

After that, the suspension of hops nanoparticles was dried using a freeze-dryer device to form hops nanoparticles. This exemplary method of encapsulating may be used with all exemplary embodiments consistent with the present disclosure.

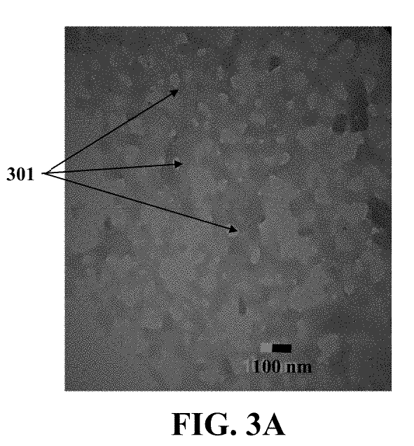

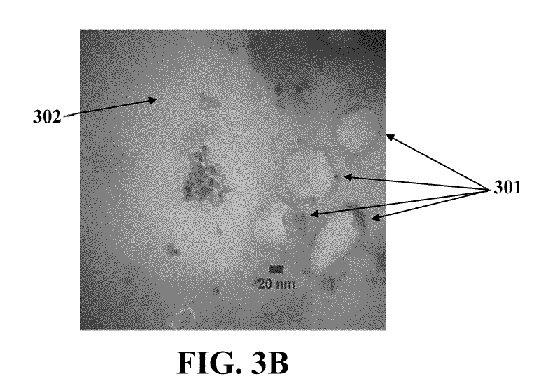

FIGS. 3A and 3B illustrate TEM images of the prepared hops nanoparticles 301 with two magnification scales. The hops nanoparticles 301 generally have a spheroidal morphology and they appear as separate and individual bodies with a uniform distribution throughout the excipient 302. Moreover, the hops nanoparticles 301 have a uniform surface without any accumulation.

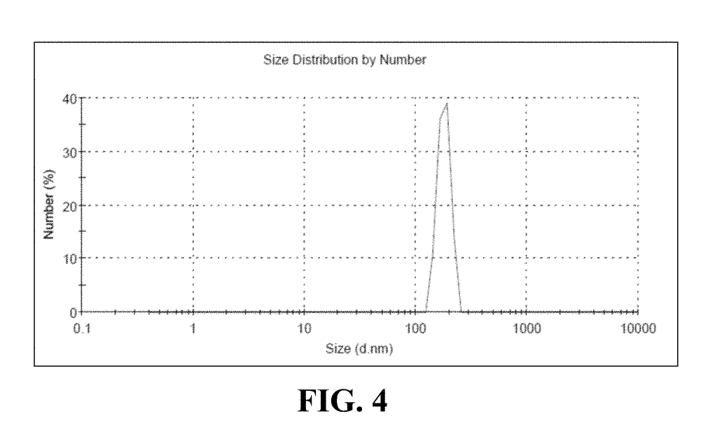

FIG. 4 illustrates size distribution curve of hops nanoparticles based on a dynamic light scattering (DLS) analysis. Referring to FIG. 4, the mean size of the hops nanoparticles is 180 nm and they have a relatively narrow size distribution. Moreover, the polydispersity index (PDI) of the hops nanoparticles is about 0.87, which indicates the monodispersity of diameter of the hops nanoparticles. In other words, diameter of the hops nanoparticles is in a uniform range which indicates the appropriate formation of the particles.

In order to obtain the topical formulation, the produced hops nanoparticles were dispersed in a mixture of an ointment-based excipient containing Vaseline and paraffin with a concentration ratio (weight/weight) of 11:89 w/w (hops nanoparticles:excipient). Dispersion of the hops nanoparticles in the mixture of the excipient was done by using a homogenizer for about 1 hour.

### Example 2: Characterization of Hops Nanoparticles

In this example, in order to study physicochemical properties of hops nanoparticles, ultraviolet-visible (UV-Vis) spectroscopy and microbial analysis were performed. A sample of hops nanoparticles, which were produced according to EXAMPLE 1, was placed in a sample site of a spectrophotometer device; and the sample was exposed to a light with a wavelength between about 200 nm and about 1100 nm. After that, the absorbance profile of the hops nanoparticles were determined, and physicochemical of the hops nanoparticles such as appearance, solubility, pH, loading, and potency were evaluated. TABLE 1 represents the physicochemical and microbial characteristics of the hops nanoparticles.

TABLE 1
Results of UV-Vis analysis of hops nanoparticles

| Test | Characteristics | Description |
|---|---|---|
| Physicochemical tests | Appearance | Pale yellow powder |
| | Solubility | Soluble |
| | PH | 5/1 |
| Active components | Loading | 26/47% |
| | Potency | 114/07% |
| Microbial analysis | Total plate count | Absent |
| | Salmonella | Absent |
| | E. Coli | Absent |
| | Yeast/Mold | Absent |

Referring to TABLE 1, the hops nanoparticles are homogenous without any air bubbles. Moreover, high potency of the hops nanoparticles indicates greater drug solubility. Referring again to TABLE 1, results of the microbial test of hops nanoparticles indicates that the sample of the hops nanoparticles does not contain any microbial contamination in terms of presence of Escherichia coli, Salmonella, yeast and fungal mold.

### Example 3: Zeta Potential of Hops Nanoparticles

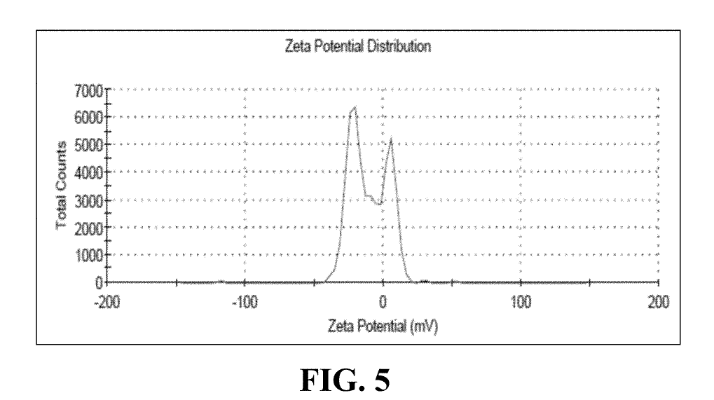

In this example, zeta potential of hops nanoparticles, which were produced according to EXAMPLE 1, were determined. FIG. 5 illustrates the zeta potential distribution curve of the hops nanoparticles. Referring to FIG. 5, the zeta potential distribution of the hops nanoparticles has three peaks, in which two of the peaks have a negative zeta potential.

TABLE 2 represents results of the zeta potential plot of FIG. 5, including zeta potential, conductivity, and mobility of the hops nanoparticles. Referring to TABLE 2, zeta potential of the hops nanoparticles is negative, −14.5 mV; and the negative zeta potential of the hops nanoparticles indicates low surface charge density and it shows their low interactions which resulted in their individual presentation of hops nanoparticles.

TABLE 2
Results of the zeta potential analysis

| Peak Number | Mean (mV) | Area (%) | Width (mV) |
|---|---|---|---|
| Peak 1: | −21.7 | 46.7 | 5.74 |
| Peak 2: | 5.19 | 31.1 | 4.64 |
| Peak 3: | −7.36 | 21.5 | 4.14 |

Results

| Parameter | Value |
|---|---|
| Zeta potential (mV) | −14.5 |
| Zeta SD (mV) | 32.6 |
| Wall Zeta Potential (mV) | −18.0 |
| Zeta Deviation (mV) | 32.6 |
| Conductivity (mS/cm) | 0.0774 |
| Effective Voltage (V) | 149 |

### Example 4: Toxicity Assay of the Topical Formulation

In this example, biocompatibility and toxicity of the topical formulation were studied. At first, cytotoxicity of the hops extract, hops nanoparticles (which were produced according to EXAMPLE 1), and β-cyclodextrin as the carrier were investigated as follows. The cultured fibroblast cells of L929 cell line were exposed to the hops extract, hops nanoparticles, and β-cyclodextrin in three test groups with different concentrations.

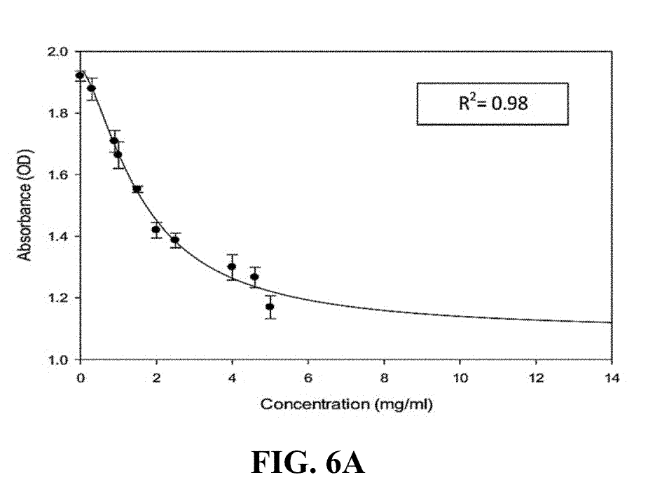

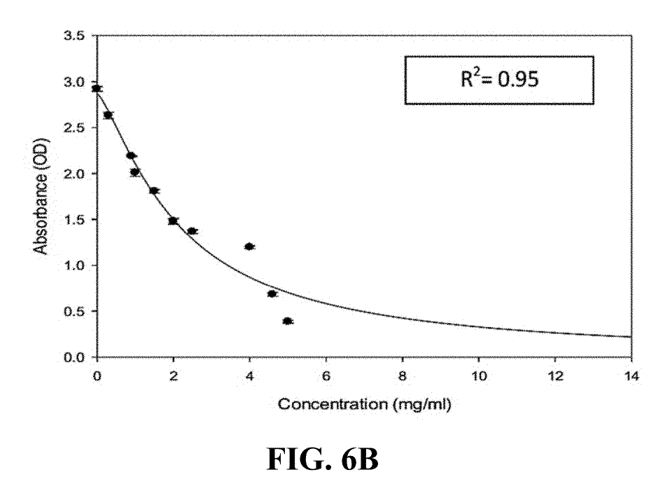

Three test groups and the control group were studied in a 3-(4,5-dimethylthiazol-2-yl)-2,5-diphenyltetra-zolium bromide (MTT) assay using dimethyl thiazolyl diphenyl tetrazolium salt for evaluating the cell toxicity. FIG. 6A illustrates a MTT plot of the fibroblast cells after about 24 hours of exposing to the hops nanoparticles. Higher absorbance of the MTT dye indicates higher cellular proliferation. Referring to FIG. 6A, after about 24 hours of exposure to the hops nanoparticles with concentrations higher than about 1 mg/ml, cellular proliferation shows a significant decrease in comparison with the cellular proliferation in the control group.

FIG. 6B illustrates a MTT plot of the fibroblast cells after about 72 hours of exposing to the hops nanoparticles. Referring to FIG. 6B, after about 72 hours of exposure to the hops nanoparticles, the cellular proliferation shows a significant decrease in all concentrations of hops nanoparticles in comparison with the cellular proliferation in the control group.

TABLE 3 represents half maximal inhibitory concentration values (IC₅₀) of the individual components of the topical formulation. Referring to TABLE 3, high IC₅₀ values of the hops nanoparticles and β-cyclodextrin as the carrier indicate that hops nanoparticles and β-cyclodextrin had no cytotoxicity effect. Therefore, the components of the topical formulation are safe products. Considering the IC₅₀ values, this drug delivery system may be beneficial for administration of drug to reduce menopausal symptoms.

TABLE 3
IC₅₀ values of the components of the topical formulation

| | IC₅₀ Value | | | |
|---|---|---|---|---|
| Component | 24 hours | | 72 hours | |
| Hops extract | 1.7% | | 0.98% | |
| Hops nanoparticles | >5 | mg/ml | 2.1 | mg/ml |
| Carrier (β-cyclodextrin) | >6 | mg/ml | >6 | mg/ml |

Moreover, in-vivo toxicity of the hops nanoparticles in the hops topical formulation were examined by topically administration of the topical formulation on the rabbit's skin. Finally, inflammatory reactions, such as erythema and edema were not observed during the toxicity test on the animal model.

### Example 5: Randomized Clinical Trial of Topical Formulation

In this example, the efficacy of the topical formulation was investigated by a randomized clinical trial (IRCT2016062526405N1) upon approval of the Biomedical Research Ethics Committee. This study was a double-blind placebo-controlled randomized trial with two groups using a placebo for a control group and a topical formulation including an ointment of hops nanoparticles for an intervention group.

The criteria for entry into the study were as follows: being between about 45 year-old and about 60 year-old, having natural menopause confirmed by amenorrhea for at least about 12 months and at most 2 years, not using hormone replacement therapy (HRT) six months prior to the study, insensitivity to herbal treatments and phytoestrogens, not having abnormal vaginal bleeding (AUB) with unknown etiology, not having breast cancer or endometrial cancer in herself or in first-degree relatives, not having cerebrovascular accident (CVA) and thromboembolism in coronary artery, not having chronic dermatological disease which needs topical protection.

Patients were also excluded if they didn't want to continue the study, had a disease during the study, and showed any allergic reactions against phytoestrogens. For conducting the study, 60 postmenopausal women who suffered from the menopausal symptoms were recruited; after that, 6 persons were excluded from the study.

Average age of the patients was about 51.2 years old, and their average age at the time of menopause was about 50.2. After informed consent, the patients were randomly allocated in two groups. The first group (intervention group) received the topical formulation including ointment of hops nanoparticles; and the second group (control group) received a placebo which was the same formulation without the hops nanoparticles.

Each patient was instructed to topically administer the topical formulation onto the arm skin, in an area with a size of about 1.5 cm length and about 1.5 cm width, once a week for about 12 consecutive weeks. In order to assess the severity of menopausal symptoms in both groups, each patient was requested to answer a menopause rating scale (MRS) questionnaire based on their menopausal symptom. Moreover, menopausal symptoms of each patient were checked at baseline and at the 12^{th} week of the study.

The MRS questionnaire includes physical, psychological, and genitourinary questions about menopausal symptoms; and lower score of MRS indicates that the patient has fewer menopausal symptoms. TABLE 4 represents scores of physical, psychological, genitourinary symptoms, and total MRS score in the intervention group and the control group.

TABLE 4
MRS score and the scores of physical, psychological,
genitourinary symptoms

| | Intervention | | Control | | | |
|---|---|---|---|---|---|---|
| Symptoms | group | | group | | | MANOVA |
| score | Mean | SD | Mean | SD | F Value | Test |
| Physical | 6.7 | 0.12 | 9.7 | 0.12 | 287.58 | P = 0.000 |
| Psychological | 6.7 | 0.19 | 10.3 | 0.19 | 154.92 | P = 0.000 |
| Genitourinary | 3.4 | 0.13 | 5.7 | 0.13 | 130.78 | P = 0.000 |
| Total MRS | 16.9 | 0.21 | 25.8 | 0.21 | 791.73 | P = 0.000 |

Referring to TABLE 4, the mean MRS score was significantly lower in the intervention group than in the control group (P<0.05). Moreover, the mean scores of physical, psychological, and genitourinary symptoms of menopause were significantly lower in the intervention group compared to the control group (P<0.05). The results indicates that the topical formulation of the hops extract is an effective medicine for reducing menopausal symptoms.

In order to assess the side effect of the topical formulation, anthropometric parameters of each patients, such as blood pressure, weight, and body mass index (BMI) were measured at baseline and at the 12^{th} week of the study. Also, patients received a checklist to record daily usage of the formulation and if they had any side effect. TABLE 5 represents anthropometric parameters in the intervention group and the control group after the 12 weeks of the study.

TABLE 5
Anthropometric parameters in the intervention group
and the control group

| | Intervention | | Control | | | |
|---|---|---|---|---|---|---|
| Anthropometric | group | | group | | F | MANOVA |
| parameter | Mean | SD | Mean | SD | Value | Test |
| Weight | 71.8 | 0.16 | 72.1 | 0.16 | 2.16 | P = 0.14 |
| BMI | 26.7 | 0.06 | 26.8 | 0.06 | 1.11 | P = 0.29 |
| Systolic blood | 122.5 | 0.8 | 120.09 | 0.8 | 3.98 | 0.052 |
| pressure | | | | | | |
| Diastolic blood | 78.9 | 0.48 | 78.6 | 0.48 | 0.14 | P = 0.71 |
| pressure | | | | | | |

Referring to TABLE 5, both of the groups had no significant differences in cases of increased weight gain, increased body mass index, or increased blood pressure (P>0.05). Moreover, no cases of uterine bleeding, increased weight gain, or hypertension were observed. In fact, results of this clinical trial confirmed the topical formulation as a safe and effective medicine for treatment of menopausal symptoms.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for reducing menopausal symptoms, the method comprising:
preparing a topical formulation of hops extract, comprising
preparing the hops extract;
encapsulating the hops extract in cyclodextrinto form hops nanoparticles; and
dispersing the hops nanoparticles in an ointment-based excipient to form the topical formulation of hops extract; and
administering the topical formulation to a part of skin.

2. The method according to claim 1, wherein dispersing the hops nanoparticles in the ointment-based excipient comprises dispersing the hops nanoparticles in the ointment-based excipient with a concentration between 10 and 15 percent by weight of the formulation.

3. The method according to claim 1, wherein preparing the hops extract comprises:
drying a plurality of hops flowers to form hops flower powder,
incubating the hops flower powder with ethanol to obtain the hops extract.

4. The method according to claim 1, wherein encapsulating the hops extract in the cyclodextrin comprises:
dissolving the hops extract powder in a protic solvent to form a hops extract solution;
dissolving the cyclodextrin in water to form a cyclodextrin solution;
mixing the hops extract solution with the cyclodextrin solution to form a suspension of hops nanoparticles; and
drying the suspension of hops nanoparticles to form hops nanoparticles.

5. The method according to claim 1, wherein the cyclodextrin comprises β-cyclodextrin matrix.

6. The method according to claim 4, wherein the protic solvent is a water-miscible solvent comprising one of ethanol, water, or combinations thereof.

7. The method according to claim 1, wherein the hops nanoparticles have an average size between 80 nm and 180 nm.

8. The method according to claim 1, wherein dispersing the hops nanoparticles includes dispersing the hops nanoparticles in the ointment-based excipient using one of a homogenizer, a stirrer, an agitator, a sonicator, an ultrasound device, or combinations thereof.

9. The method according to claim 1, wherein the ointment-based excipient includes one of Vaseline, or paraffin, or combinations thereof.

10. The method according to claim 1, wherein administering the topical formulation to a part of skin comprises topically administering the topical formulation with a therapeutically effective amount of the hops nanoparticles, wherein the therapeutically effective amount of the hops nanoparticles is between 30 mg/day and 90 mg/day.

11. The method according to claim 1, wherein the menopausal symptoms includes one of hot flushes, night sweats, sleep disturbances, fatigue, anxiety, sexual problems, and dryness of vagina, and combinations thereof.

12. A topical formulation for reducing menopausal symptoms, comprising:
a plurality of hops nanoparticles, comprising:
hops extract encapsulated in cyclodextrin; and
an ointment-based excipient,
wherein the hops nanoparticles with a concentration between 5 and 15 percent by weight of the formulation are dispersed within the ointment-based excipient.

13. The topical formulation according to claim 12, wherein the hops nanoparticles have an average size between 80 nm and 180 nm.

14. The topical formulation according to claim 12, wherein the hops nanoparticles has a pH between 5 and 7.

15. The topical formulation according to claim 12, wherein the ointment-based excipient includes one of Vaseline, or paraffin, or combinations thereof.

16. The topical formulation according to claim 12, wherein the cyclodextrin comprises β-cyclodextrin matrix.

17. The topical formulation according to claim 12, wherein the formulation is topically administered onto a part of skin with a therapeutically effective amount of the hops nanoparticles, wherein the therapeutically effective amount of the hops nanoparticles is between 30 mg/day and 90 mg/day.

18. The topical formulation according to claim 12, wherein the menopausal symptoms includes one of hot flushes, night sweats, sleep disturbances, fatigue, anxiety, sexual problems, and dryness of vagina, and combinations thereof.
